(19) United States
(12) Patent Application Publication
(10) Pub. No.: US 2006/0167051 A1
(43) Pub. Date: Jul. 27, 2006

(54) Crystalline non-solvated 1-(4-(2-piperidinylethoxy)phenoxy)-2-(4-methanesulfonylphenyl)-6-hydroxynaphthalene hydrochloride

(76) Inventor: David Michael Remick, Fishers, IN (US)

Correspondence Address: ELI LILLY & COMPANY, PATENT DIVISION, P.O. BOX 6288, INDIANAPOLIS, IN, 46206-6288, US

(21) Appl. No.: 10/542,872

(22) PCT Filed: Jan. 21, 2004
(86) PCT No.: PCT/US04/00020

§ 371 Date: Jul. 20, 2005

Related U.S. Application Data
(60) Provisional application No. 60/450,233, filed on Feb. 25, 2003.

(51) Int. Cl. C07D 211/06; A61K 31/445

(52) U.S. Cl. 514/319; 546/206

(57) ABSTRACT

The present invention relates to crystalline non-solvated 1-(4-2-piperidinylethoxy)phenoxy)-2-(4-methane-sulfonylphenyl)-6-hydroxynaphthalene hydrochloride, useful as a selective estrogen receptor modulator.

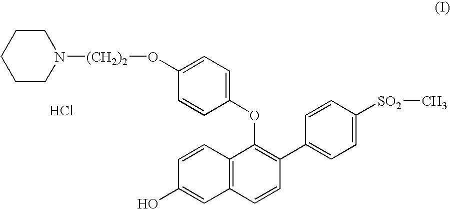

This application claims priority to U.S. Provisional Patent Application No. 60/450,233, filed Feb. 25, 2003 and International (WO) Patent Application No.

PCT/IB03/03349, filed Jul. 16, 2003.

### BACKGROUND OF THE INVENTION

Uterine leiomyoma/leiomyomata (uterine fibroid disease) is a clinical problem that goes under a variety of names, including uterine fibrosis, uterine hypertrophy, uterine leiomyomata, myometrial hypertrophy, fibrosis uteri, and fibrotic metritis. Essentially, uterine fibrosis is a condition where there is an inappropriate deposition of fibroid tissue on the wall of the uterus. This condition is a cause of dysmenorrhea and infertility in women.

Endometriosis is a condition of severe dysmenorrhea, which is accompanied by severe pain, bleeding into the endometrial masses or peritoneal cavity and often leads to infertility. The symptoms' cause appears to be ectopic endometrial growths that respond inappropriately to normal hormonal control and are located in inappropriate tissues. Because of the inappropriate locations for endometrial growth, the tissue seems to initiate local inflammatory-like responses causing macrophage infiltration and a cascade of events leading to initiation of the painful response. Evidence suggests that a cause of uterine fibrosis and endometriosis is an inappropriate response of fibroid tissue and/or endometrial tissue to estrogen.

Many publications have appeared within the last ten years disclosing selective estrogen receptor modulators (SERMs), e.g., U.S. Pat. Nos. 5,484,795, 5,484,798, 5,510,358, 5,998,401 and WO 96/09040. Many of these SERMs, generally speaking, have been found to have a beneficial estrogen agonist activity in the bone and cardiovascular systems with a concomitant beneficial estrogen antagonist activity in the breast. A small, particularly useful subset of such compounds has also been found to have an estrogen antagonist effect in the uterus. A compound with this SERM profile holds particular promise in treating uterine fibroid disease and/or endometriosis.

However, the clinical use of such SERM compounds for the treatment of uterine fibroid disease and/or endometriosis, particularly in pre-menopausal women, has been hampered by the propensity of said compounds to have significant ovarian stimulatory effects. A great need currently exists, therefore, for new SERM compounds that behave as estrogen antagonists in the uterus that do not significantly stimulate the ovaries.

### SUMMARY OF INVENTION

The present invention relates to crystalline non-solvated 1-(4-(2-piperidinylethoxy)phenoxy)-2-(4-methanesulfonylphenyl)-6-hydroxynaphthalene hydrochloride, that is, a compound of the formula: 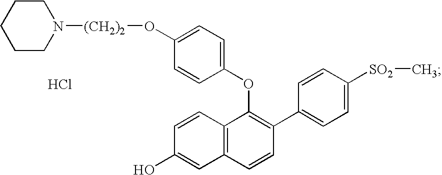
hereafter referred to as F-III; having an X-ray diffraction pattern which comprises the following peaks: 15.2±0.1, 17.6±0.1, 18.6±0.1 and 24.1±0.1° in 2θ; when the pattern is obtained from a copper radiation source (CuKα, λ=1.54056 Å).

The present invention also relates to a pharmaceutical formulation containing F-III and a pharmaceutical carrier. In another embodiment, the pharmaceutical formulations of the present invention may be adapted for use in treating endometriosis and/or uterine leiomyoma.

The present invention also relates to methods for treating endometriosis and/or uterine leiomyoma which comprise administering to a patient in need thereof an effective amount of F-III.

In addition, the present invention relates to F-III for use in treating endometriosis and/or uterine leiomyoma. The present invention is further related to the use of F-III for the manufacture of a medicament for treating endometriosis and/or uterine leiomyoma.

### BRIEF DESCRIPTION OF THE FIGURE

FIG. 1 is a representative XRD pattern for F-III.

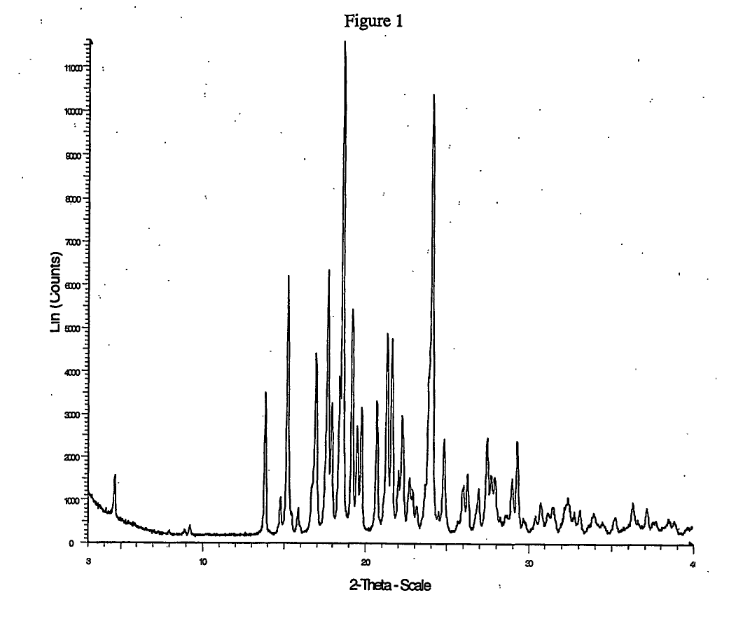

### DETAILED DESCRIPTION OF THE INVENTION

Being a non-solvated crystal form, it should be understood that F-III is an anhydrous form of 1-(4-(2-piperidinylethoxy)phenoxy)-2-(4-methanesulfonylphenyl)-6-hydroxynaphthalene hydrochloride.

X-ray powder diffraction (XRD) was used to characterize F-III. XRD is a technique that detects long-range order in a crystalline material. X-ray powder diffraction (XRD) patterns were obtained on a Siemens D5000 X-ray powder diffractometer, equipped with a CuKα source (λ=1.54056 Å) and a Kevex solid state Si(Li) detector operated at minimally 50 kV and 40 mA. The samples were scanned from 4 to 35° in 2θ, with a step size of 0.02° in 2θ and a maximum scan rate of 3.0 seconds per step and with 1 mm divergence and receiving slits and a 0.1 mm detector slit. Synthetic fluorophlogopite mica (NIST 675) was used as an internal standard to correct any sample displacement errors.

The XRD patterns for F-III disclosed herein feature sharp peaks and a flat baseline, indicative of highly crystalline materials. The angular peak positions in 2θ and corresponding I/Iₒ data for all peaks with intensities equal to or greater than 10% of the largest peak for F-III are shown in Table 1. All data in Table 1 is expressed with an accuracy of ±0.1° in 2θ.

TABLE 1
F-III

| Angle 2θ | I/Iₒ (%) |
|---|---|
| 4.6 | 13.2 |
| 13.8 | 30.1 |
| 15.2 | 53.2 |
| 16.9 | 37.9 |
| 17.6 | 54.5 |
| 17.9 | 27.8 |
| 18.3 | 25.9 |
| 18.6 | 100.0 |
| 19.2 | 46.8 |
| 19.5 | 23.4 |
| 19.7 | 27.0 |
| 20.7 | 28.3 |
| 21.3 | 41.9 |
| 21.6 | 40.8 |
| 22.0 | 14.4 |
| 22.2 | 25.3 |
| 22.7 | 13.0 |
| 23.8 | 33.4 |
| 24.1 | 89.5 |
| 24.8 | 20.8 |
| 26.0 | 11.1 |
| 26.3 | 13.7 |
| 26.9 | 10.9 |
| 27.4 | 21.0 |
| 27.9 | 13.2 |
| 29.0 | 12.9 |
| 29.3 | 20.3 |

It is well known in the crystallography art that, for any given crystal form, the relativeintensities of the diffraction peaks may vary due to preferred orientation resulting from factors such as crystal morphology and habit. Where the effects of preferred orientation are present, peak intensities are altered, but the characteristic peak positions of the polymorph are unchanged. See, e.g., The United States Pharmacopeia #23, National Formulary #18, pages 1843-1844, 1995. Furthermore, it is also well known in the crystallography art that, for any given crystal form, the angular peak positions may vary slightly. For example, peak positions can shift due to a variation in the temperature at which a sample is analyzed, sample displacement, or the presence or absence of an internal standard. In the present case, a peak position variability of ±0.1° in 2θ will take into account these potential variations without hindering the unequivocal identification of the crystalline salts of the present invention.

¹³C Cross polarization/magic angle spinning (CP/MAS) NMR (solid-state NMR or SSNMR) was also used to characterize F-III and. Said spectrum was obtained using a Varian Unity Inova 400 MHz NMR spectrometer operating at a carbon frequency of 100.573 MHz. Acquisition parameters were as follows: 90° proton r.f. pulse width 4.0 μs, contact time 2.5 ms, pulse repetition time 15 s, MAS frequency 10 kHz, spectral width 50 kHz, and acquisition time 50 ms. Chemical shifts were referenced to the methyl group of hexamethylbenzene (δ=17.3 ppm) by sample replacement. The chemical shift data on Form III is as follows: 20.7, 23.8, 25.3, 38.4, 51.2, 52.4, 56.8, 59.8, 110.8, 113.3, 114.6, 118.0, 118.3, 122.3, 122.9, 125.4, 127.2, 130.0, 132.0, 137.9, 142.2, 143.1, 147.3, 151.3, 153.8, and 158.0 ppm.

### Preparation 1

### 1-(2-{4-[2-(4-methanesulfonyl-phenyl)-6-methoxy-naphthalen-1-yloxy]-phenoxy}-ethyl)-piperidine hydrochloride

To a 3-neck flask equipped with a reflux condenser and nitrogen vent purge, add 6-methoxytetralone (1.0 eq.), 4-bromophenyl-methyl-sulfone (1.02˜1.05 eq.), palladium acetate Pd(OAc)₂ (0.025 eq.), [(Oxydi-2,1-phenylene) bis(dipheniylphosphine)] (DPEphos ligand, 0.026 eq.) and toluene 10˜12 volumes. Add sodium t-butoxide (2.5 eq.) in one portion and allow mixture to exotherm to ˜40° C. Heat to 75° to 80° C. Upon reaction completion, as judged by HPLC analysis, cool to room temperature. Add 12 volumes water slowly keeping the temperature <40° C. Stir 2 to 3 hours. Filter over polypropylene pad and wash with water (3×2 volumes). Dry the filter cake overnight at 50° C. to give 2-(4-methanesulfonylphenyl)-6-methoxytetralone.

Combine 2-(4-methanesulfonylphenyl)-6-methoxytetralone (1.0 eq.), hyflo (20%/weight), and toluene (7.5 volumes). Add PBr₃ (1.5˜1.75 eq.) in one portion while stirring at room temperature. Heat contents to reflux (˜110° C.) overnight. Upon reaction completion, as judged by HPLC analysis (usually 15 hours), cool solution to 45° C. or 90° C. and slowly add 20 volumes tetrahydrofuran (THF). Stir for 30 minutes at 45° or 90° C. and filter warm over a pad of hyflo. Wash the pad with 2×2 volumes THF at 45° C. or 90° C. Concentrate filtrate to approximately 7 volumes. Add 7.5 volumes water to the remaining mixture keeping the temperature below 40° C. (NOTE: initial addition of water is very exothermic with large evolution of HBr). Cool slurry to room temperature and stir for 2 to 3 hours. Filter over a polypropylene pad and wash with 2×2 volumes water. Dry filter cake overnight at 60° C. under vacuum to give 1-bromo-2-(4-methanesulfonylphenyl)-3,4-dihydro-6-methoxynaphthalene.

Combine 1-bromo-2-(4-methanesulfonylphenyl)-3,4-dihydro-6-methoxynaphthalene and 2,3-dichloro-5,6-dicyano-1,4-benzoquinone (DDQ, 1.8 equiv.) in 10 volumes acetonitrile and 5 volumes of THF. Under nitrogen atmosphere, heat reaction contents to 73-75° C. Monitor reaction progress by GC analysis until reaction completion. Additional DDQ (0.2-0.3 equiv.) may be required for reaction completion. Cool contents to ambient temperature and add 10 volumes 1 N sodium hydroxide. Stir for approximately 1 hour and filter. Rinse filter cake with 2 volumes water, 3×5 volumes 50% acetonitrile/water and fmally 3 volumes methanol. Vacuum dry the filter cake at 65° C. to give 1-bromo-2-(4-methanesulfonylphenyl)-6-methoxynaphthalene.

Charge 1-bromo-2-(4-methanesulfonylphenyl)-6-methoxynaphthalene, 4-(2-piperidinylethoxy)phenol (2.0 equiv), cesium carbonate (2.0-2.1 equiv.) and copper chloride (0.15 equiv.) to 12 volumes of diglyme. Evacuate flask for ˜2 minutes, then purge with nitrogen. Repeat evacuation/nitrogen purge 3 times. Heat the contents to 130° C. until reaction completion as judged by HPLC analysis. Upon reaction completion, cool contents to near ambient temperature and add 12 volumes of ammonium hydroxide and stir for approximately 30 minutes. Filter to remove solids and wash solids with 9 volumes of 30% MeOH/NH₄OH, slurrying the solids on the filter support. Wash solids with 2×9 volumes of 30% NH₄OH/MeOH, slurrying solids on filter support. Wash with 4 volumes methanol. Vacuum dry filter cake at 60° C. to give the free base of the title compound. Slurry the free base in 9 volumes of toluene and heat the slurry to 70-75° C. Dissolve 1.1 equivalents of hydrogen chloride gas in 2 volumes of ethanol. Add the ethanolic HCl solution to the hot toluene slurry. Cool solution to ambient temperature and stir 1-2 hours. Filter and wash with a small amount of toluene. Vacuum dry the filter cake at 65° C. to give the title compound.

### EXAMPLE 1

### F-III

Combine 1-(2-{4-[2-(4-methanesulfonyl-phenyl)-6-methoxy-naphthalen-1-yloxy]-phenoxy}-ethyl)-piperidine hydrochloride with 5 volumes 1,2-dichloroethane (DCE) and cool the mixture to <10° C. Add by subsurface addition 5 equivalents of boron trichloride. Stir at ambient temperature until reaction is complete, as judged by HPLC analysis. Quench reaction contents into 5.6 volumes 3A-ethanol (ethanol denatured with about 5% methanol) keeping the contents <50° C. Cool to ambient temperature and stir for 1-3 hours. Filter the solids and rinse the filter cake with 3A-ethanol. Vacuum dry the filter cake at 65° C. Dissolve the isolated product in 9.8 volumes of 3A ethanol and 1.5 volumes of deionized water at about the reflux temperature of the mixture. Allow the solution to reflux for approx 30 minutes then allow the mixture to cool to ambient temperature. Once at ambient temperature, allow the resultant slurry to stir for 1-2 hours at ambient temperature, then filter and rinse the filter cake with 3A ethanol. Dissolve the filter cake in 19 volumes of acetonitrile and 1.4 volumes of deionized water at reflux. Azeotropically remove the water by distillation until a total of 12.1 volumes of distillate are removed. Cool the resulting slurry to ambient temperature, filter and rinse the filter cake with acetonitrile to give the title compound

Formulation (Pharmaceutical Composition)

The compound of the present invention is preferably formulated in a dosage unit form, i.e., in an individual delivery vehicle, for example, a tablet or capsule, prior to administration to the recipient patient. The term “patient” includes female humans and non-human female animals such as companion animals (dogs, cats, horses and the like). The preferred patient of treatment is a female human.

The present pharmaceutical compositions are prepared by known procedures using well-known and readily available ingredients. In making the formulations of the present invention, the active ingredient (F-III) will usually be mixed with a carrier, or diluted by a carrier, or enclosed within a carrier which may be in the form of a capsule, sachet, paper or other container. When the carrier serves as a diluent, it may be a solid, semisolid or liquid material that acts as a vehicle, excipient or medium for the active ingredient. Thus, the compositions can be in the form of tablets, pills, powders, lozenges, sachets, cachets, elixirs, suspensions, emulsions, solutions, syrups, aerosol (as a solid or in a liquid medium), soft and hard gelatin capsules, suppositories, sterile injectable solutions and sterile packaged powders.

Some examples of suitable carriers, excipients, and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methyl and propylbydroxybenzoates, talc, magnesium stearate and mineral oil. The formulations can additionally include lubricating agents, wetting agents, e.g., polysorbate 80 or lauryl sulfate,.emulsifying and suspending agents, preserving agents, sweetening agents or flavoring agents. The compositions of the invention may be formulated so as to provide quick, sustained or delayed release of the active ingredient after administration to the recipient patient.

### FORMULATION EXAMPLES

### 10 mg Capsules or Tablets

Add about 156 mg of a bulking agent (lactose, mannitol, or dextrose), about 20 mg of a disintegrant (microcrystalline cellulose, or starch), about 4 mg of a super disintegrant (crospovidone, or sodium starch glycollate) about 4 mg of a binder (hydroxy propyl methyl cellulose or hydroxy propyl cellulose) and about 10 mg of F-III to a granulator and mix to uniformly distribute the powders. Spray an aqueous granulation solution consisting of povidone, hydroxy propyl methyl cellulose, or hydroxy propyl cellulose (sufficient to deliver about 2-4% by weight of dry powders) and wetting agent such as polysorbate 80 or sodium lauryl sulfate (sufficient to deliver between 0.5 and 3% by weight) at a uniform rate onto the powders while mixing. Wet sieve the granulated material through a screen to disrupt large agglomerates. Dry the filtered granulated powder by either fluid bed processing or in a convection oven. Reduce the dried granulated powder to a uniform size by passing through a co-mill or other suitable apparatus and then transfer the material to a mixer. Uniformly blend the granulated powder with a lubricant (magnesium stearate, or sodium stearyl fumurate at about 1% by weight of the total formulation) and additional disintegrant (about 2-4% by weight in the outside powders). Fill the finished powders into hard gelatin capsules or compress said powder into tablets (followed by film coating the tablets as described below). The total weight of a capsule or tablet prepared in this manner is about 200 mg.

### 45 mg Capsules or Tablets

Add about 162 mg of a bulking agent (lactose, mannitol or starch), about 10 mg of a disintegrant (crospovidone or sodium starch glycollate), and about 45 mg of F-III to a granulator and mix to uniformly distribute the powders. Spray an aqueous granulation solution consisting of povidone (about 35% by weight) and polysorbate 80 (about 10% by weight) at a uniform rate onto the powders while mixing. Wet sieve the granulated material through a screen to disrupt large agglomerates. Dry the filtered granulated powder by either fluid bed processing or in a convection oven. Pass the dried granulated powder through a co-mill or other suitable apparatus and then transfer the material to a mixer. Uniformly blend the granulated powder with a lubricant (magnesium stearate; about 1% by weight of the total formulation) and additional disintegrant (about 2% in the outside powders). Fill the finished powders into hard gelatin capsules or compress said powder into tablets (followed by film coating the tablets as described below). The total weight of a capsule or tablet prepared in this manner is about 230 mg.

Alternatively, to prepare a tablet, add the bulking agent, disintegrant, and F-III to a mixer and blend to uniformly distribute the powders. Once the powders are uniformly distributed, add the lubricant and blend again. Transfer the blended material to a tablet compression machine to prepare the tablets which are subsequently film coated with an appropriate film forming agent.

Biological Assays

Ishikawa Cell Proliferation Assay: This assay measures cell proliferation (using an alkaline phosphatase readout) in both an agonist mode in the presence of a compound of the present invention alone, and in an antagonist mode in which the ability of a compound of the present invention to block estradiol stimulation of growth is measured.

Ishikawa human endometrial tumor cells are maintained in MEM (minimum essential medium, with Earle's salts and L-Glutamine, Gibco BRL, Gaithersburg, Md.), supplemented with 10% fetal bovine serum (FBS) (V/V), (Gibco BRL). One day prior to assay, growth media is changed to assay medium, DMEM/F-12 (3:1) (Dulbecco's Modified Eagle Medium: Nutrient Mixture F-12, 3:1 Mixture, phenol red-free, Gibco BRL) supplemented with 5% dextran coated charcoal stripped fetal bovine serum (DCC-FBS) (Hyclone, Logen, Utah), L-Glutamine (2 mM), MEM sodium pyruvate (1 mM), HEPES (N-[2-hydroxyethyl]piperazine-N′-[2-ethanesulfonic acid] 2 mM) all from Gibco BRL). After an overnight incubation, Ishikawa cells are rinsed with Dulbecco's Phosphate Buffered Saline (1×) (D-PBS) without Ca⁺² and Mg⁺² (Gibco BRL), and trypsinized by a 3 minute incubation with 0.25% Trypsin/EDTA, phenol red-free (Gibco BRL). Cells are resuspended in assay medium and adjusted to 250,000 cells/mL. Approximately 25,000 cells in a 100 ul media are added to flat-bottom 96 wells microculture plates (Costar 3596) and incubated at 37° C. in a 5% CO₂ humidified incubator for 24 hours. The next day, serial dilutions of compounds are prepared in assay medium (at 6 times the final concentration in the assay). The assay is run in dual mode, agonist and antagonist modes.

For the agonist mode, plates receive 25 μl/well of assay medium followed by 25 μl/well of a diluted compound of the present invention (at 6× the final concentrations). For the antagonist mode, plates receive 25 μl/well of 6 nM E₂ (β-Estradiol, Sigma, St. Louis, Mo.) followed by 25 μl/well of a diluted compound of the present invention (at 6× the final concentrations). After an additional 48-hour incubation at 37° C. in a 5% CO₂ humidified incubator, media is aspirated from wells and 100 μl fresh assay medium is added to each microculture. Serial dilutions of compounds are prepared and added to the cells as described above. After an additional 72 hour incubation at 37° C. in a 5% CO₂ humidified incubator, the assay is quenched by removing media and rinsing plates twice in Dulbecco's Phosphate Buffered Saline (1×) (D-PBS) (Gibco BRL). The plates are dried for 5 minutes and frozen at −70° C. for at least 1 hour. The plates are then removed from the freezer and allowed to thaw at room temperature. To each well, 100 μl of 1-Step™ PNPP (Pierce Chemical Company, Rockford, Ill.) is added. After a 20-minute incubation, plates are read on a spectophotometer at 405 nm.

The data is fitted to a linear interpolation to derive EC50 (for agonist mode) or IC50 (for antagonist mode) values. For the antagonist mode, a % efficacy for each compound is calculated versus E2 (1 nM) alone. For the agonist mode, a % efficacy for each compound is calculated versus the response to tamoxifen.

In the agonist mode, F-III is tested and is less stimulatory than tamoxifen. In the antagonist mode, F-III inhibits greater than at least 70% of the 1 nM estradiol response.

10-Day Rat Hormone (Ovarian Stimulation) Screen: An initial, first screen for ovarian toxicity is conducted using a 10-day rat hormone study to measure estradiol and luteinizing hormone levels after GYN SERM F-III administration. This screen is conducted by administering compound by oral gavage for 10 days to mature (9-10 week old) F344 female rats. Trunk blood is collected by rapid decapitation for evaluation of LH and estradiol levels approximately 2 hours after the 10^{th} dose. Serum, obtained by centrifugation, is removed and stored frozen below −60° C. until assayed. Serum levels of LH and estradiol are measured using radioimmunoassay (RIA) methods.

Rat LH primary antibody and reference preparations (rat LH:RP-3) are obtained from Dr. A. F. Parlow, Director, Pituitary Hormones and Antisera Center, Harbor-UCLA Medical Center, Torrance, Calif. The LH assay upper limits of detection were 30 ng/mL and the lower limits of detection were 0.1 ng/mL for the 100 μl samples.

E2 Clinical Assays. DiaSorin s.r.l., Saluggia (Vercelli), Italy. The upper limit of detection is 1000 pg/mL and the lower limit of detection is 5 ng/mL. F-III is tested in the above assay and does not significantly elevate circulating estradiol or LH levels.

Utilities

As an antagonist of estrogen in breast and uterine tissue, F-III is useful in treating conditions in which estrogen has been demonstrated to play a causal role therein. As an agonist of estrogen in skeletal and cardiovascular systems, F-III is useful in treating conditions in which estrogen has been demonstrated to play a beneficial role therein.

The terms “treating” and “treat” as used herein, include their generally accepted meanings, i.e., alleviating, ameliorating, managing, preventing, prohibiting, restraining, slowing, stopping, or reversing the progression or severity of a pathological condition, or sequela thereof, described herein. The term “preventing” refers to reducing the likelihood that the recipient of a compound of the present invention will incur or develop any of the pathological conditions, or sequela thereof, described herein.

The diseases, disorders or conditions for which a compound of the present invention is useful in treating include, but are not limited to, (1) uterine and/or breast cancer; (2) endometriosis; (3) uterine leiomyoma/leiomyomata; and (4) osteoporosis. Treatment of uterine leiomyoma/leiomyomata as described herein, may also reduce associated symptoms such as pain, urinary frequency, and uterine bleeding.

Dose

As used herein, the term “effective amount” means an amount of F-III that is capable of treating conditions, or detrimental effects thereof, described herein.

The specific dose administered is determined by the particular circumstances surrounding each situation. These circumstances include, the route of administration, the prior medical history of the recipient, the pathological condition or symptom being treated, the severity of the condition/symptom being treated, and the age and sex of the recipient. The recipient patient's physician should determine the therapeutic dose administered in light of the relevant circumstances.

Generally, an effective minimum daily dose of F-III will exceed about 5 mg. Typically, an effective maximum daily dose will not exceed about 350 mg. The exact dose may be determined, in accordance with the standard practice in the medical arts of “dose titrating” the recipient; that is, initially administering a low dose of the compound, and gradually increasing the dose until the desired therapeutic effect is observed.

Route of Administration

F-III may be administered by a variety of routes including the intramuscular, intranasal, intravaginal, intravenous, oral, rectal, subcutaneous, topical and transdermal routes. A preferred route of administration is the oral route.

Combination Therapy

F-III may be used in combination with other drugs that are used in the treatment of the diseases or conditions for which these compounds are useful. Such other drug(s) may be administered, by a route and in an amount commonly used therefore, contemporaneously or sequentially with a salt of the present invention. When F-III is used contemporaneously with one or more other drugs, a pharmaceutical unit dosage form containing such other drugs in addition to the present compound is preferred. Accordingly, the pharmaceutical compositions of the present invention include those that contain one or more other active ingredients. One example of another other active ingredient that may be combined with a compound of the present invention, either administered separately or in the same pharmaceutical composition, includes agents employed in hormone replacement therapy (HRT).

1. Crystalline non-solvated 1-(4-(2-piperidinylethoxy)phenoxy)-2-(4-methanesulfonylphenyl)-6-hydroxynaphthalene hydrochloride having an X-ray diffraction pattern which comprises the following peaks: 15.2±0.1, 17.6±0.1, 18.6±0.1 and 24.1±0.10 in 2θ; when the pattern is obtained from a copper radiation source (CuKα, λ=1.54056 Å).

2. The salt of claim 2 wherein said X-ray diffraction pattern further comprises the following peaks: 16.9±0.1, 19.2±0.1, 21.3±0.1 and 21.6±0.10 in 2θ.

3. A method of treating endometriosis comprising administering to a patient in need thereof an effective amount of a compound of claim 1.

4. A method of treating uterine leiomyoma comprising administering to a patient in need thereof an effective amount of a compound of claim 1.

5. (canceled)

6. A method of treating endometriosis comprising administering to a patient in need thereof an effective amount of a compound of claim 2.

7. A method of treating uterine leiomyoma comprising administering to a patient in need thereof an effective amount of a compound of claim 2.
